(19) United States
(12) Patent Application Publication
(10) Pub. No.: US 2026/0174332 A1
(43) Pub. Date: Jun. 25, 2026

(54) NON-INVASIVE BLOOD VISCOSITY MEASUREMENT METHOD AND SYSTEM BASED ON OXYGEN SATURATION AND BLOOD FLOW FOR PREDIABETES PREDICTION AND DEHYDRATION MEASUREMENT

(71) Applicant: UIF (UNIVERSITY INDUSTRY FOUNDATION), YONSEI UNIVERSITY, Seoul (KR)

(72) Inventor: Sungwoo LEE, Seoul (KR)

(21) Appl. No.: 19/126,986

(22) PCT Filed: Dec. 9, 2022
(86) PCT No.: PCT/KR2022/020071

§ 371 Date: Jun. 12, 2025

(30) Foreign Application Priority Data
Nov. 3, 2022 (KR) 10-2022-0144937

(51) Int. Cl. A61B 5/00; A61B 5/1455; A61B 8/06

(52) U.S. Cl. CPC A61B 5/0059; A61B 5/1455; A61B 5/14551; A61B 5/14552; A61B 5/7207; A61B 8/06

(57) ABSTRACT

Provided is a method and a system for noninvasive measurement of blood viscosity based on oxygen saturation and blood flow rate, which may easily and quickly calculate the blood viscosity by using the method and the system for noninvasive measurement of blood viscosity based on oxygen saturation and blood flow rate, thereby enabling immediate obtaining of the blood viscosity.

### TECHNICAL FIELD

The present invention relates to a method and a system for measuring blood viscosity, and more particularly, to a method and a system for noninvasive measurement of blood viscosity, which may easily and quickly calculate the blood viscosity based on oxygen saturation and blood flow rate.

### BACKGROUND ART

Blood viscosity refers to an internal resistance that occurs when blood flows in a blood vessel of the human body. The blood viscosity varies depending on blood flow velocity, blood vessel diameter, and blood pressure, and therefore has been noted as an indicator that may comprehensively interpret the blood flow. The blood viscosity has been found to be related to cardiovascular and cerebrovascular diseases through prior studies. Hypertension, diabetes, and hyperlipidemia caused by the cardiovascular and cerebrovascular diseases may cause some parts of the blood vessel to narrow, thus increasing the blood viscosity, thereby damaging vascular endothelium and causing abnormal blood flow, which may lead to the cerebrovascular disease.

Therefore, various systems and methods for measuring blood viscosity have been developed to assess a risk of blood circulation diseases by measuring the blood viscosity. Previously, Japanese Patent No. 3785084 discloses a system for measuring vascular endothelial function that may measure quantitative indicators of vascular endothelial function.

The prior system for testing vascular function performs calculation by using the Navier-Stokes equation. However, the prior system requires a large amount of calculation when calculating a measured value while measuring information on the blood vessel or blood flow that varies based on shear stress. Accordingly, it requires a long time and involves complexity in calculating the measured value of the blood vessel or blood flow that varies based on the shear stress.

### DISCLOSURE

### Technical Problem

The present invention is invented to solve at least some of the problems of the prior art as described above, and an object of the present invention is to provide a method and a system for noninvasive measurement of blood viscosity based on oxygen saturation and blood flow rate for the noninvasive measurement of blood viscosity.

### Technical Solution

In one general aspect, provided is a method for noninvasive measurement of blood viscosity, the method including: a emission step of emitting light into a blood vessel; a blood vessel information obtaining step of receiving light reflected from the blood vessel and obtaining the image information and biological information of the blood vessel; and a first information calculation step of calculating oxygen saturation and hematocrit based on the image information and biological information of the blood vessel obtained in the blood vessel information obtaining step; a second information calculation step of calculating the hematocrit and a blood shear rate based on the calculated first information; and a blood viscosity calculation step of calculating the blood viscosity based on the second information.

In another general aspect, provided is a system for noninvasive measurement of blood viscosity, the system including: a light emission unit for emitting light into a blood vessel; a blood vessel information obtaining unit for receiving light reflected from the blood vessel and obtaining the image information and biological information of the blood vessel; and a blood viscosity calculation unit for calculating first information used for calculating oxygen saturation and hematocrit based on the image information and biological information of the blood vessel obtained in the blood vessel information obtaining step, for calculating second information used for calculating the hematocrit and a blood shear rate based on the calculated first information, and for calculating the blood viscosity based on the second information.

### Advantageous Effects

The method and the system for noninvasive measurement of blood viscosity based on oxygen saturation and blood flow rate according to the present invention may derive the relationship equation between the oxygen saturation and hematocrit based on the relationship between the blood flow rate and the blood shear rate and the relationship between the hematocrit and blood viscosity, and may easily and quickly obtain the blood viscosity by substituting the relationship equation between the oxygen saturation and the hematocrit into the relationship equation between the blood shear rate and the blood viscosity.

In addition, the system according to the present invention may include the measurement system mounted on a finger and the wearable system on a wrist, and may be included in the alarm system capable of immediately alerting the user when the rapid change occurs in the blood viscosity.

### DESCRIPTION OF DRAWINGS

FIG. 1 is a schematic diagram showing a relationship between diabetes and blood viscosity.

FIG. 2 is a graph showing a relationship between hematocrit and the blood viscosity.

FIG. 3 is a graph showing a relationship equation between a blood shear rate and the blood viscosity.

FIG. 4 is a flow chart showing a method for noninvasive measurement of blood viscosity based on oxygen saturation and blood flow rate according to the present invention.

FIG. 5 is a block diagram showing a system for noninvasive measurement of blood viscosity based on oxygen saturation and blood flow rate according to the present invention.

FIG. 6 is a schematic diagram showing the system for noninvasive measurement of blood viscosity that includes a wearable system.

### BEST MODE

In one general aspect, provided is a method for noninvasive measurement of blood viscosity, the method including: a light emission step of emitting light into a blood vessel; a blood vessel information obtaining step of receiving light reflected from the blood vessel and obtaining the image information and biological information of the blood vessel; and a first information calculation step of calculating oxygen saturation and hematocrit based on the image information and biological information of the blood vessel obtained in the blood vessel information obtaining step; a second information calculation step of calculating the hematocrit and a blood shear rate based on the calculated first information; and a blood viscosity calculation step of calculating the blood viscosity based on the second information.

In the first information calculation step, the first information may be used to calculate a blood flow rate based on incident light having two wavelengths λ₁ and λ₂ and incident on the blood vessel.

In the second information calculation step, the oxygen saturation may be calculated based on an intensity ratio of incident light, which is determined according to a ratio of an alternating current (AC) signal to a direct current (DC) signal, obtained from incident light having two wavelengths λ₁ and λ₂ and incident on the blood vessel, and the ratio of the AC signal to the DC signal, obtained from incident light having two wavelengths and incident on the blood vessel.

In the second information calculation step, the blood shear rate may be calculated based on the oxygen saturation, which is calculated in the first information calculation step, obtained from incident light having two wavelengths λ₁ and λ₂ and incident on the blood vessel.

In the blood viscosity calculation step, the blood viscosity may be calculated based on the blood shear rate and the hematocrit obtained in the first information calculation step and the second information calculation step, respectively.

The method may further include: a derivation step of deriving a relationship equation among the oxygen saturation, the blood shear rate, and the hematocrit after the blood viscosity calculation step; and a computation step of computing the blood viscosity by computing a relationship equation between the oxygen saturation and the blood shear rate and a relationship equation between the oxygen saturation and the hematocrit, which are derived from the derivation step.

In the light emission step of emitting light into the blood vessel, incident light having two different wavelengths may be emitted.

In another general aspect, provided is a system for noninvasive measurement of blood viscosity, the system including: a light emission unit for emitting light into a blood vessel; a blood vessel information obtaining unit for receiving light reflected from the blood vessel and obtaining the image information and biological information of the blood vessel; and a blood viscosity calculation unit for calculating first information used for calculating oxygen saturation and hematocrit based on the image information and biological information of the blood vessel obtained in the blood vessel information obtaining step, for calculating second information used for calculating the hematocrit and a blood shear rate based on the calculated first information, and for calculating the blood viscosity based on the second information.

The light emission unit may emit incident light having two different wavelengths.

The first information may be used to calculate a blood flow rate based on incident light having two wavelengths λ₁ and λ₂ and incident on the blood vessel, the second information may be used to calculate the hematocrit based on the oxygen saturation, which is calculated in the first information calculation step, obtained from incident light having two wavelengths λ₁ and λ₂ and incident on the blood vessel, and a relationship equation between an alternating current (AC) signal and a direct current (DC) signal, obtained from incident light having two wavelengths and incident on the blood vessel, and the blood viscosity may be calculated based on the hematocrit and the blood shear rate obtained from the first information and the second information, respectively.

The system may include a wearable system, may be connected to a wireless communication system to detect an amount of change in the blood viscosity, and may be included in an alarm system capable of immediately alerting a user when a rapid change occurs in the blood viscosity.

Hereinafter, the present invention is described in more detail with reference to an embodiment or a specific embodiment including the accompanying drawings. However, the embodiment or specific embodiment described below is provided only as an example for describing the present invention in detail. The present invention is not limited thereto and may be implemented in various forms.

Unless otherwise defined, all technical and scientific terms may have the same meanings as those commonly understood by any one of those skilled in the art to which the present invention pertains. Terms used in the present invention are provided only to effectively describe the specific embodiment, and are not intended to limit the present invention.

In addition, a term of a single number as used in the specification and the appended claims is intended to include its plural number unless the context clearly indicates otherwise. In addition, unless explicitly described to the contrary, “including” any components will be understood to imply the inclusion of other components rather than the exclusion of any other components.

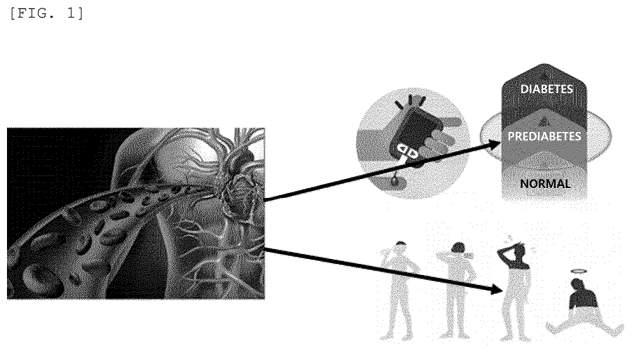

The present invention relates to a method and a system for noninvasive measurement of blood viscosity based on oxygen saturation and blood flow rate, and FIG. 1 is a schematic diagram showing a relationship between diabetes and blood viscosity.

Referring to FIG. 1, it may be seen that high blood viscosity may damage the vascular endothelium, thus increasing a risk of thrombosis. Diabetes may increase a risk of myocardial infarction, stroke, limb amputation, or the like, and cardiovascular disease is one of the most important causes of death in diabetic patients. Therefore, the blood viscosity is a factor that needs to be addressed primarily in a progression of diabetes and vascular disease. Therefore, the method and the system for noninvasive measurement of blood viscosity based on oxygen saturation and blood flow rate according to the present invention may measure the blood viscosity in real time in a noninvasive manner, thereby enabling immediate identification of physiological abnormality information such as a likelihood of diabetes development and dehydration symptoms, which are related to the blood viscosity.

The description describes the blood flow rate, the blood shear rate, the hematocrit, and the blood viscosity before describing the method and the system for noninvasive measurement of blood viscosity based on oxygen saturation and blood flow rate according to the present invention. The blood flow rate may be obtained by multiplying a blood area A by a blood flow velocity V, and the blood shear rate may be obtained by dividing the blood flow velocity V by a blood vessel length L. Therefore, a value of the blood shear rate (unit: 1/s) may be calculated by dividing the measured blood flow rate by a blood vessel volume (unit: m³).

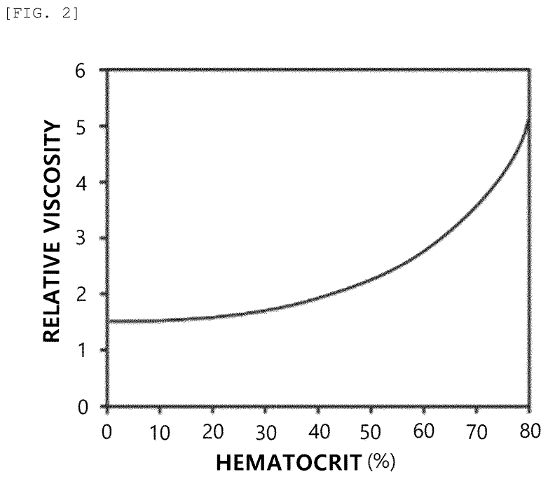

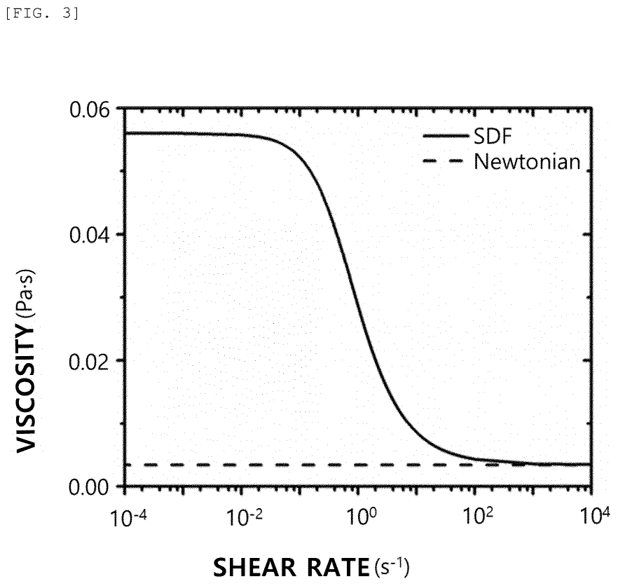

In addition, FIG. 2 is a graph showing a relationship between the hematocrit and the blood viscosity, and FIG. 3 is a graph showing a relationship equation between the blood shear rate and the blood viscosity. Referring to FIGS. 2 and 3, it may be determined that the hematocrit and the blood viscosity are proportional upon determining the relationship between the hematocrit and the blood viscosity before obtaining the blood viscosity based on the oxygen saturation.

The description describes the method for noninvasive measurement of blood viscosity based on oxygen saturation and blood flow rate by using relationships among the blood flow rate, the blood shear rate, the hematocrit, and the blood viscosity described above.

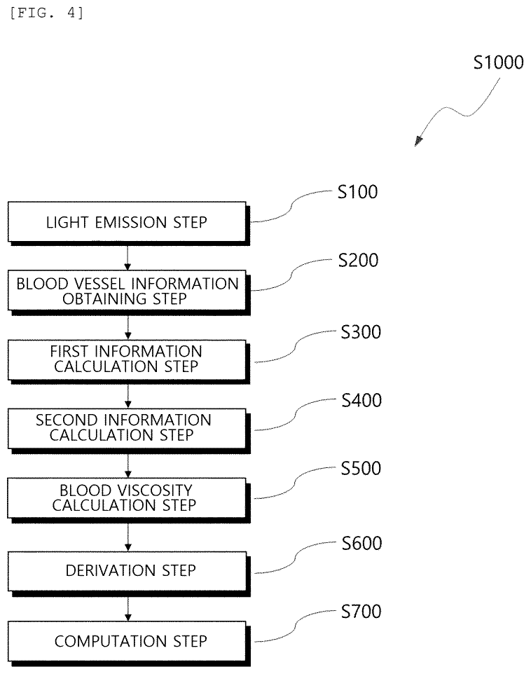

FIG. 4 is a flow chart showing the method for noninvasive measurement of blood viscosity based on oxygen saturation and blood flow rate according to the present invention. Referring to FIG. 4, a method (S1000) for noninvasive measurement of blood viscosity based on oxygen saturation and blood flow rate according to the present invention may include a light emission step (S100), a blood vessel information obtaining step (S200), a first information calculation step (S300), a second information calculation step (S400), a blood viscosity calculation step (S500), a derivation step (S600), and a computation step (S700).

The light emission step (S100) refers to a step of emitting light into a blood vessel. Here, a light emission unit may emit incident light having two different wavelengths. Specifically, a path length of light incident on the blood vessel is unable to be precisely determined based on the oxygen saturation, and incident light having two different wavelengths may thus be emitted into the blood vessel.

The blood vessel information obtaining step (S200) refers to a step of receiving light reflected from the blood vessel and obtaining the image information and biological information of the blood vessel. In the blood vessel information obtaining step (S200), the image information and biological information of the blood vessel may be obtained using light emitted into the blood vessel in the light emission step (S100).

The first information calculation step (S300) refers to a step of calculating the oxygen saturation and the blood flow rate based on the image information and biological information of the blood vessel obtained in the blood vessel information obtaining step. The first information may be used to calculate the hematocrit based on the oxygen saturation obtained from incident light having two wavelengths λ₁ and λ₂ and incident on the blood vessel, and the blood flow rate may be calculated based on the calculated hematocrit.

The second information calculation step (S400) refers to a step of calculating the hematocrit and the blood shear rate based on the calculated first information. The oxygen saturation may be calculated based on incident light having two different wavelengths λ₁ and λ₂, and R, which indicates a ratio of an alternating current (AC) signal to a direct current (DC) signal. Here, in the light emission step (S100), light emitted to the blood vessel may exhibit the DC signal having an irregular signal when passing through the skin, muscles, bones, or the like, and may exhibit the AC signal having a regular signal when light emitted through a skin tissue and the blood vessel passes through arterial blood or the like. Therefore, the oxygen saturation may be calculated by using the two wavelengths λ₁ and λ₂ and the value R, which indicates the ratio of the AC signal to the DC signal. Specifically, the oxygen saturation may be calculated based on an intensity ratio of incident light, which is determined according to a ratio of the AC signal to the DC signal, obtained from incident light having two wavelengths λ₁ and λ₂ and incident on the blood vessel, and the ratio of the AC signal to the DC signal obtained from incident light having two wavelengths λ₁ and λ₂ and incident on the blood vessel. More specifically, in the first information calculation step, the oxygen saturation may be calculated based on a relationship between incident light having two wavelengths λ₁ and λ₂ and incident on the blood vessel and a relationship equation between the AC signal and the DC signal obtained from incident light having two wavelengths and incident on the blood vessel, thereby calculating the hematocrit. Here, incident light having two wavelengths and incident on the blood vessel refers to light having two wavelengths that exhibit the most different variation characteristics with respect to the hematocrit. Here, an amount of change in a blood vessel thickness may vary from person to person, and it is therefore difficult to precisely measure hemoglobin concentration. Therefore, it is necessary to minimize the difference in the amount of change in the blood vessel thickness that varies from person to person, and to set the maximum range of the amount of change based on variation in the hematocrit. The two different wavelengths are the wavelengths that exhibit the most different variation characteristics with respect to the hematocrit, and the hematocrit may therefore be calculated using a ratio of the two different wavelengths.

The blood viscosity calculation step (S500) refers to a step of calculating the blood viscosity based on the blood shear rate and the hematocrit obtained in the first information calculation step and the second information calculation step, respectively. Specifically, the blood viscosity may be calculated based on the hematocrit and the blood shear rate obtained in the first information calculation step and the second information calculation step, respectively. More specifically, the blood viscosity may be obtained by substituting the hematocrit into the blood shear rate, and by substituting the oxygen saturation, which is calculated from incident light having two different wavelengths λ₁ and λ₂ and R, which indicates the ratio of the AC signal to the DC signal, into the hematocrit. In addition, the second information may be calculated by substituting the oxygen saturation, which is calculated in the first information calculation step, obtained from incident light having two wavelengths λ₁ and λ₂ and incident on the blood vessel, into the ratio of the blood viscosity to the blood shear rate, calculated in the second information calculation step (S400), thereby obtaining the hematocrit. Specifically, the blood viscosity may be obtained based on the blood shear rate. The blood shear rate may be obtained by dividing a difference between the blood viscosity when the blood shear rate is zero and the blood viscosity when the blood shear rate is infinite by the blood shear rate.

The derivation step (S600) refers to a step of deriving a relationship equation among the oxygen saturation, the blood shear rate, and the hematocrit. The value R, which indicates the ratio of the AC signal to the DC signal obtained from incident light having two different wavelengths λ₁ and λ₂ with respect to the oxygen saturation may be the same as the ratio of the two wavelengths with respect to the hematocrit, and the relationship equation between the oxygen saturation and the hematocrit may thus be derived by using R, which is a common value for the oxygen saturation and the hematocrit. In addition, the relationship equation between the oxygen saturation and the blood shear rate may be derived by using R, which is the common value for the oxygen saturation and the hematocrit, and H, which is a hematocrit value.

The computation step (S700) refers to a step of computing the blood viscosity by computing the relationship equation between the oxygen saturation and the blood shear rate and the relationship equation between the oxygen saturation and the hematocrit, which are derived from the derivation step (S600). The blood viscosity may be computed by using R, which is the common value for the oxygen saturation and the hematocrit derived from the derivation step (S600), and H, which is the hematocrit value, into the blood shear rate.

The relationship equation between the oxygen saturation and the hematocrit and the relationship equation between the oxygen saturation and the blood shear rate may use a universal constant that does not reflect a patient's accumulated values for constants for experimentally determinable constants. However, it is difficult to perform precise calculation because the blood shear rate and the hematocrit differ from person to person. Therefore, the method for noninvasive measurement of blood viscosity based on oxygen saturation and blood flow rate according to the present invention may obtain the blood viscosity based on the accumulated values of the blood shear rate and the hematocrit values of the patient. Accordingly, the amount of change in the blood viscosity for each patient may be easily and precisely measured.

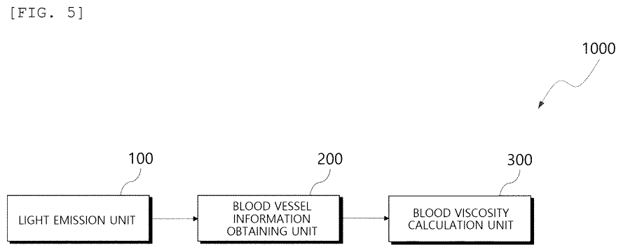

Next, FIG. 5 is a block diagram showing the system for noninvasive measurement of blood viscosity based on oxygen saturation and blood flow rate according to the present invention. Referring to FIG. 5, a system 1000 for noninvasive measurement of blood viscosity based on oxygen saturation and blood flow rate according to the present invention may include a light emission unit 100, a blood vessel information obtaining unit 200, and a blood viscosity calculation unit 300. The light emission unit 100 may emit light into the blood vessel. Specifically, the light emission unit 100 may emit incident light having two different wavelengths. Here, incident light having two different wavelengths may be emitted to precisely determine the path length of light incident on the blood vessel.

The blood vessel information obtaining unit 200 may receive light emitted by the light emission unit and then reflected from the blood vessel to thus obtain the image information and biological information of the blood vessel.

The blood viscosity calculation unit 300 may calculate the first information used for calculating the oxygen saturation and the hematocrit based on the image information and biological information of the blood vessel obtained in the blood vessel information obtaining step, calculate the second information used for calculating the oxygen saturation and the blood shear rate based on the calculated first information, and calculate the blood viscosity based on the second information. Specifically, the first information may be used to calculate the hematocrit based on the oxygen saturation obtained from incident light having two wavelengths λ₁ and λ₂ and incident on the blood vessel and the relationship equation between the alternating current signal and the direct current signal, obtained from incident light having two wavelengths and incident on the blood vessel. In addition, the second information may be used to calculate the hematocrit and the blood shear rate based on the oxygen saturation, which is calculated in the first information calculation step, obtained from incident light having two wavelengths λ₁ and λ₂ and incident on the blood vessel. In addition, the blood viscosity may be calculated based on the hematocrit and the blood shear rate obtained from the first information and the second information, respectively.

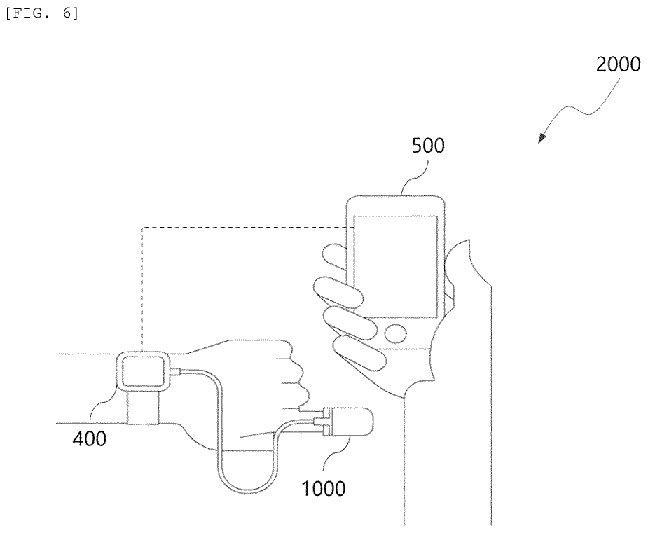

FIG. 6 is a schematic diagram showing the system for noninvasive measurement of blood viscosity that includes a wearable system. Referring to FIG. 6, the system for noninvasive measurement of blood viscosity may include a measurement system mounted on a finger and the wearable system mounted on a wrist, may be connected to a system such as a smartphone to detect an amount of change in the blood viscosity, and may be included in an alarm system capable of immediately alerting a user when a rapid change occurs in the blood viscosity.

Therefore, the method and the system for noninvasive measurement of blood viscosity based on oxygen saturation and blood flow rate according to the present invention may derive the relationship equation between the oxygen saturation and the hematocrit based on the relationship between the blood flow rate and the blood shear rate and the relationship between the hematocrit and the blood viscosity, and easily and quickly obtain the blood viscosity by substituting the relationship equation between the oxygen saturation and the hematocrit into the relationship equation between the blood shear rate and the blood viscosity.

In addition, the system may include the measurement system mounted on a finger and the wearable system mounted on a wrist, and may thus be included in the alarm system capable of immediately alerting the user when the rapid change occurs in the blood viscosity.

The present invention is not limited to the above-described embodiments, may be variously applied, and may be variously modified without departing from the gist of the present invention claimed in the appended claims.

### INDUSTRIAL APPLICABILITY

A method and a system for noninvasive measurement of blood viscosity based on oxygen saturation and blood flow rate for prediabetes prediction and dehydration measurement according to the present invention may provide a technology for deriving a relationship equation between the oxygen saturation and hematocrit, based on a relationship between the blood flow rate and a blood shear rate and a relationship between the hematocrit and the blood viscosity, and for easily and quickly obtaining the blood viscosity by substituting a relationship equation between the oxygen saturation and the hematocrit into a relationship equation between the blood shear rate and the blood viscosity. Therefore, the present invention may be used in an alarm system capable of immediately alerting a user when a rapid change occurs in the blood viscosity.

1. A method for noninvasive measurement of blood viscosity, the method comprising:
a light emission step of emitting light into a blood vessel;
a blood vessel information obtaining step of receiving light reflected from the blood vessel and obtaining the image information and biological information of the blood vessel; and
a first information calculation step of calculating oxygen saturation and hematocrit based on the image information and biological information of the blood vessel obtained in the blood vessel information obtaining step;
a second information calculation step of calculating the hematocrit and a blood shear rate based on the calculated first information; and
a blood viscosity calculation step of calculating the blood viscosity based on the second information.

2. The method of claim 1, wherein in the first information calculation step, the calculated first information is used to calculate a blood flow rate based on incident light having two wavelengths λ₁ and λ₂ and incident on the blood vessel.

3. The method of claim 1, wherein in the second information calculation step,
the oxygen saturation is calculated based on an intensity ratio of incident light, which is determined according to a ratio of an alternating current (AC) signal to a direct current (DC) signal, obtained from incident light having two wavelengths λ₁ and λ₂ and incident on the blood vessel, and the ratio of the AC signal to the DC signal, obtained from incident light having two wavelengths and incident on the blood vessel.

4. The method of claim 1, wherein in the second information calculation step,
the blood shear rate is calculated based on the oxygen saturation, which is calculated in the first information calculation step, obtained from incident light having two wavelengths λ₁ and λ₂ and incident on the blood vessel.

5. The method of claim 1, wherein in the blood viscosity calculation step,
the blood viscosity is calculated based on the blood shear rate and the hematocrit obtained in the first information calculation step and the second information calculation step, respectively.

6. The method of claim 1, further comprising:
a derivation step of deriving a relationship equation among the oxygen saturation, the blood shear rate, and the hematocrit after the blood viscosity calculation step; and
a computation step of computing the blood viscosity by computing a relationship equation between the oxygen saturation and the blood shear rate and a relationship equation between the oxygen saturation and the hematocrit, which are derived from the derivation step.

7. The method of claim 1, wherein in the light emission step of emitting light into the blood vessel,
incident light having two different wavelengths is emitted.

8. A system for noninvasive measurement of blood viscosity, the system comprising:
a light emission unit for emitting light into a blood vessel;
a blood vessel information obtaining unit for receiving light reflected from the blood vessel and obtaining the image information and biological information of the blood vessel; and
a blood viscosity calculation unit for calculating first information used for calculating oxygen saturation and hematocrit based on the image information and biological information of the blood vessel obtained in the blood vessel information obtaining step, for calculating second information used for calculating the hematocrit and a blood shear rate based on the calculated first information, and for calculating the blood viscosity based on the second information.

9. The system of claim 8, wherein the light emission unit emits incident light having two different wavelengths.

10. The system of claim 8, wherein the first information is used to calculate a blood flow rate based on incident light having two wavelengths λ₁ and λ₂ and incident on the blood vessel,
the second information is used to calculate the hematocrit based on the oxygen saturation, which is calculated in the first information calculation step, obtained from incident light having two wavelengths λ₁ and λ₂ and incident on the blood vessel, and a relationship equation between an alternating current (AC) signal and a direct current (DC) signal, obtained from incident light having two wavelengths and incident on the blood vessel, and
the blood viscosity is calculated based on the hematocrit and the blood shear rate obtained from the first information and the second information, respectively.

11. The system of claim 8, wherein the system includes a wearable system,
is connected to a wireless communication system to detect an amount of change in the blood viscosity, and
is included in an alarm system capable of immediately alerting a user when a rapid change occurs in the blood viscosity.
